(19) United States
(12) Patent Application Publication
(10) Pub. No.: US 2025/0298547 A1
(43) Pub. Date: Sep. 25, 2025

(54) IMAGE FORMING APPARATUS, IMAGE FORMING METHOD, AND STORAGE MEDIUM

(71) Applicant: CANON KABUSHIKI KAISHA, Tokyo (JP)

(72) Inventor: TATEKI NARITA, Tokyo (JP)

(21) Appl. No.: 19/083,118

(22) Filed: Mar. 18, 2025

(30) Foreign Application Priority Data
Mar. 19, 2024 (JP) 2024-043877

(51) Int. Cl. G06F 3/12; G16H 40/20; H04N 1/00

(52) U.S. Cl. CPC G06F 3/1204; G06F 3/1243; G06F 3/1288; G16H 40/20; H04N 1/00212; H04N 1/00233; H04N 1/00244; H04N 1/00331

(57) ABSTRACT

An image forming apparatus includes a printer, at least one processor, and at least one memory. The at least one memory stores instructions for causing the at least one processor and the at least one memory to receive image data posted to a chat service and identification information on a user who has posted the image data, and output print data by compositing the received image data and information corresponding to the identification information with a form image. The printer is configured to print an image on a sheet based on the output print data.

### BACKGROUND

### Field of the Disclosure

The present disclosure relates to an image forming apparatus, an image forming method, and a storage medium.

### Description of the Related Art

In order to create a patient registration card with a face photograph thereon used in a hospital or a similar facility for each patient, print data (patient registration card form) in which information from the hospital is written is prepared in advance, and print data to which variable information (patient's name and face photograph) is added is created and printed by a printer at the time of issuing the patient registration card for the corresponding patient.

Japanese Patent Application Laid-Open No. 2007-245470 discloses a technique of receiving print data and form designation information, reading corresponding form information, and setting whether to composite the form information with the print data.

For example, when a patient has a patient registration card with a face photograph thereon issued, the patient provides photograph data to the hospital, and a hospital clerk creates print data by adding the photograph data to a patient registration card form in which fixed information from the hospital is written and printing the print data by a printer. It thus takes time and effort for a hospital to perform the issuing procedure.

### SUMMARY

Embodiments of the present disclosure are directed to reducing time and effort when creating print data in which image data is composited with a form.

In view of the forgoing issue, according to an aspect of the present disclosure, an image forming apparatus includes a printer, at least one processor, and at least one memory. The at least one memory stores instructions for causing the at least one processor and the at least one memory to receive image data posted to a chat service and identification information on a user who has posted the image data, and output print data by compositing the received image data and information corresponding to the identification information with a form image. The printer is configured to print an image on a sheet based on the output print data.

Further features of various embodiments will become apparent from the following description of exemplary embodiments with reference to the attached drawings.

### BRIEF DESCRIPTION OF THE DRAWINGS

FIG. 1 is a diagram illustrating an example of a system configuration in a hospital reception operation.

FIG. 2 is a diagram illustrating an example of a hardware configuration of an image processing apparatus.

FIG. 3 is a diagram illustrating an example of a hardware configuration of a user terminal.

FIG. 4 is a diagram illustrating an example of a hardware configuration of a chat tool server.

FIG. 5 is a diagram illustrating an example of a process of image compositing and printing from a chat tool.

FIG. 6 is a diagram illustrating an example of a patient intake form.

FIG. 7 is a diagram illustrating an example of a patient registration card form.

FIG. 8 is a diagram illustrating an example of form data information.

FIG. 9 is a diagram illustrating an association of identification information and user information.

FIG. 10 is a diagram illustrating an example of chat messages.

FIG. 11 is a diagram illustrating an example of message events between the chat tool server and the image processing apparatus.

FIG. 12 is a sequence diagram illustrating an example of an image compositing and printing from the chat tool.

FIG. 13 is a flowchart illustrating an example of an image compositing and printing process from the chat tool.

FIG. 14 is a flowchart illustrating an example of an image compositing and printing process from the chat tool.

FIG. 15 is a diagram illustrating an example of a user information registration request screen.

### DESCRIPTION OF THE EMBODIMENTS

Hereinafter, example embodiments for carrying out the present disclosure will be described with reference to the drawings. Programs for a control unit 110 according to flowcharts for an image processing apparatus are stored in a read-only memory (ROM) 112 or a storage 114 of the control unit 110, loaded to a random access memory (RAM) 113, and executed by a central processing unit (CPU) 111. In the flowcharts for an image processing apparatus, programs, which are stored in the ROM 112, are read to the RAM 113 and executed by the CPU 111.

A first exemplary embodiment will be described in a case where a photograph of a pet as a face photograph is composited with a patient registration card in an animal hospital.

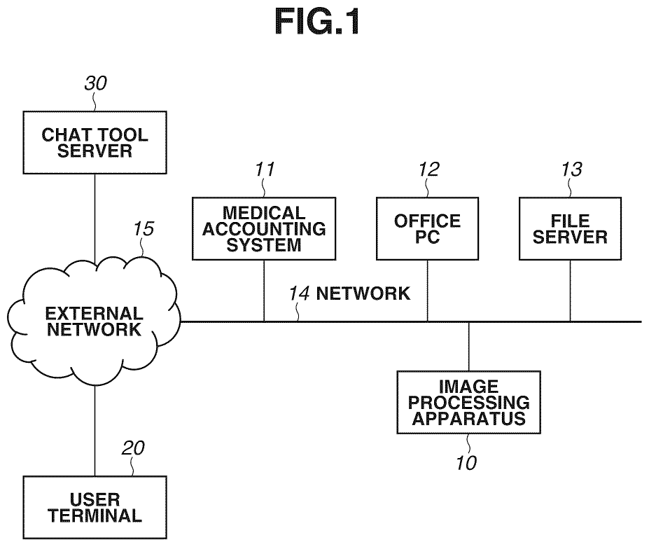

FIG. 1 is a diagram illustrating an example of a system configuration in a hospital reception operation.

In the present exemplary embodiment, an image processing apparatus 10, a medical accounting system 11, an office personal computer (PC) 12, and a file server 13 are connected to each other via a network 14 and are all communicable with each other. The network 14, a user terminal 20, and a chat tool server 30 are connected to each other via an external network 15, and are all communicable with each other. The medical accounting system 11 is a system for performing administrative tasks and accounting processing at a hospital desk, and is used with the office PC 12. The file server 13 stores data used in the hospital reception operation and is used on the office PC 12. The image processing apparatus 10, which also serves as an image forming apparatus, prints received document data or transmits scanned document data to another device. The user terminal 20 is an information terminal, such as a smartphone, a tablet terminal, or a personal computer, used by a user of a chat tool. While FIG. 1 illustrates an example in which one terminal is connected, any number of terminals connected can be used. A user operates the user terminal 20 to access the chat tool server 30 and exchange messages with another user or other users. The user terminal 20 and the chat tool server 30, which are connected to a wired or wireless communication network, can transmit and receive data to and from each other. In the present exemplary embodiment, a public network on the Internet is assumed as an example of the communication network, but a dedicated line may be used. The chat tool server 30 is a chat server arranged on the Internet and a server that provides a chat service using a chat tool 306. The chat tool server 30 executes processes related to exchange of messages, such as a process for transmitting and receiving, for example, messages, to and from the user terminal 20, and display of a display screen or display screens for message transmission and reception. The chat tool server 30 can perform processes on exchange of messages and the like in groups each consisting of three or more members. Further, the chat tool server 30 provides a bot service, which allows an automatic response process of responding to a message including a specific keyword with a predetermined message and a webhook process of hooking a received message to a set Uniform Resource Locator (URL) via Hyper Transfer Protocol (HTTP) communication.

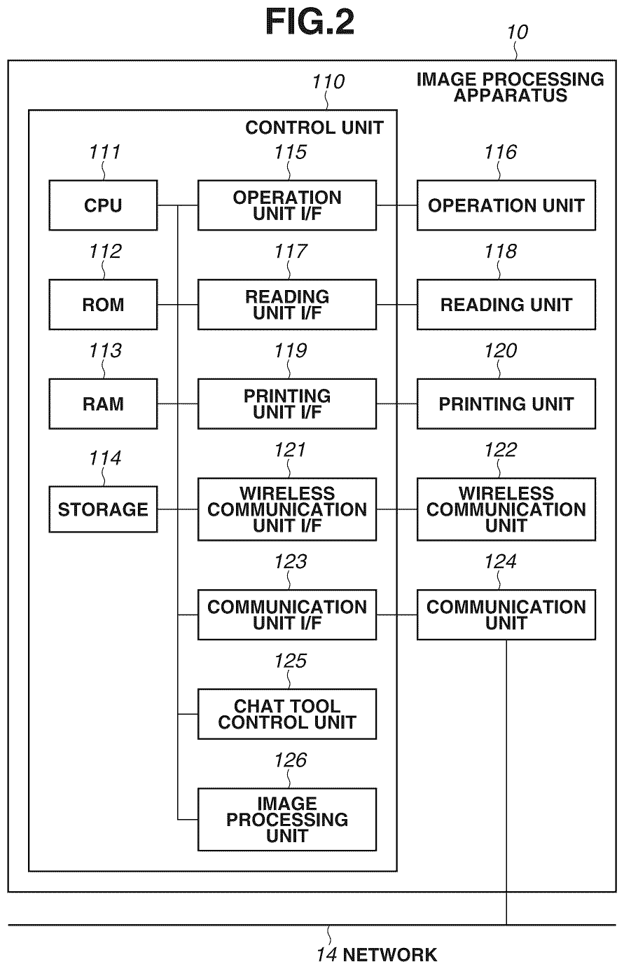

FIG. 2 is a diagram illustrating an example of a hardware configuration of the image processing apparatus 10.

The image processing apparatus 10 includes the CPU 111, the ROM 112, the RAM 113, the storage 114, an operation unit interface (I/F) 115, and an operation unit 116. The image processing apparatus 10 further includes a reading unit I/F 117, a reading unit 118, a printing unit I/F 119, a printing unit 120, a wireless communication unit I/F 121, a wireless communication unit 122, a communication unit I/F 123, and a communication unit 124. The control unit 110 including the CPU 111 generally controls the operation of the image processing apparatus 10. The CPU 111 loads control programs stored in the ROM 112 or the storage 114 to the RAM 113 and performs various kinds of control, such as reading control and printing control. The ROM 112 stores control programs executable by the CPU 111. The ROM 112 also stores boot programs, font data, and the like. The RAM 113 is a main storage memory and is used as a working area and a temporary storage area for loading various kinds of control programs stored in the ROM 112 and the storage 114. The storage 114 stores image data, print data, various kinds of programs, various kinds of addresses, and various kinds of setting information. In the present exemplary embodiment, a flash memory is assumed as the storage 114, but an auxiliary storage device, such as a solid state drive (SSD) or a hard disk drive (HDD) may be used. Further, an embedded MultiMediaCard (eMMC™) may be used. In the image processing apparatus 10 according to the present exemplary embodiment, one CPU 111 executes each process illustrated in flowcharts described below using one memory (RAM 113), but some embodiments are not limited thereto. For example, pluralities of CPUs, RAM units, ROM units, and storage devices may be operated in cooperation to execute each process illustrated in the flowcharts described below. Further, a process may be partially executed by using a hardware circuit, such as an Application-Specific Integrated Circuit (ASIC) or a Field-Programmable Gate Array (FPGA). The operation unit I/F 115 connects the control unit 110 and the operation unit 116 including a display unit, such as a touch panel, and hard keys, to each other. The operation unit 116 displays information to a user and detects an input from the user. The reading unit I/F 117 connects the reading unit 118, such as a scanner, and the control unit 110 to each other. The reading unit 118 reads an image of a document, and the CPU 111 generates image data, such as binary data, from the image. The image data generated based on the image read by the reading unit 118 is transmitted to an external device or printed on a recording paper sheet. The printing unit I/F 119 connects the printing unit 120, such as a printer, and the control unit 110 to each other. The CPU 111 transfers image data (print data) stored in the RAM 113 to the printing unit 120 via the printing unit I/F 119. The printing unit 120 prints an image based on the transferred image data on a recording paper sheet fed from a sheet feed cassette. The wireless communication unit I/F 121 is an interface for controlling the wireless communication unit 122, and wirelessly connects to an external wireless device via the control unit 110. The user terminal 20 may be used as the external wireless device. The communication unit I/F 123 connects the control unit 110 and the network 14. The communication unit I/F 123 is used by the communication unit 124 to transmit image data and various kinds of information in the image processing apparatus 10 to an external device on the network 14, and to receive print data from an information processing apparatus on the network 14 and information in the network 14. A method of transmission and reception via the network 14 includes transmission and reception using electronic mails (e-mails) or file transmission using other protocols (e.g., File Transfer Protocol (FTP), Server Message Block (SMB), and Web Distributed Authoring and Versioning (WebDAV)).

A chat tool control unit 125 can transmit and receive image data and various kinds of data via the network 14 by the chat tool server 30 accessing the chat tool control unit 125 via the communication unit I/F 123 in HTTP communication using message Application Programming Interfaces (APIs) of the chat tool server 30.

An image processing unit 126 includes a plurality of ASICs that perform image processes, such as rendering (raster image conversion), resolution conversion, compression/decompression, binary-to-multilevel conversion, or compositing layout, on input and output image data. For example, the image processing unit 126 can generate image data for printing by rendering document data received via the communication unit I/F 123, and composite and lay out a plurality of pieces of image data input as an image of one page via the reading unit I/F 117. The image processing apparatus 10, which has a form compositing and printing function, can generate image data for printing in which other pieces of image data and text data are composited and laid out at predetermined positions with respect to document data or image data specified as form data.

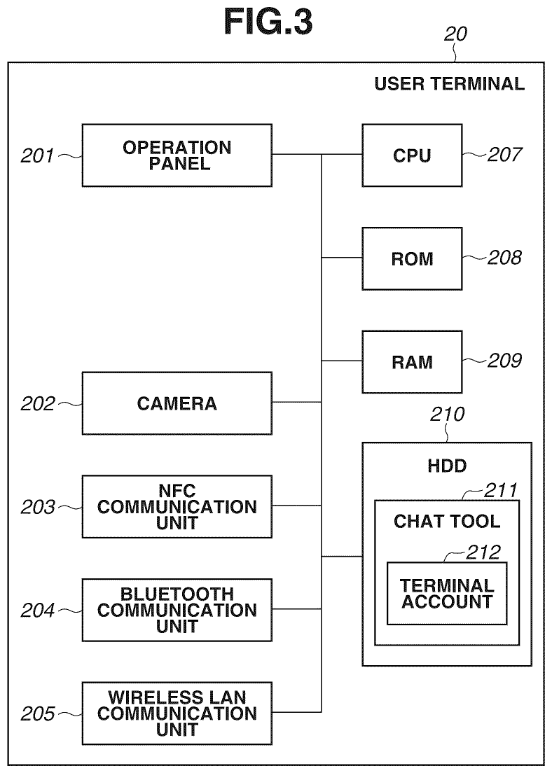

FIG. 3 is a diagram illustrating an example of a hardware configuration of the user terminal 20.

The user terminal 20 according to the present exemplary embodiment is assumed to be an apparatus, such as a smartphone or a tablet PC, but may be another type of apparatus as long as the apparatus is an information processing apparatus capable of Wi-Fi® communication.

A CPU 207 reads control programs stored in a ROM 208 and executes various processes for controlling the operation of the user terminal 20. The ROM 208 stores the control programs. A RAM 209 is used as a temporary storage area, such as a main memory or a work area of the CPU 207. An HDD 210 stores various kinds of data, such as photographs and electronic documents. An operation panel 201 has a touch panel function capable of detecting touch operations by users, and displays various kinds of screen provided by an OS or an e-mail transmission application. For example, a chat tool 211 provided by the chat tool server 30 is installed as an application on the HDD 210. In addition, on the chat tool 211, a terminal account 212 of the user terminal 20 issued by the chat tool server 30 is created, and information stored in the chat tool 211 is displayed on the operation panel 201. A user can input a desired operation instruction to the user terminal 20 by inputting a touch operation to the operation panel 201. The user terminal 20 includes not-illustrated hardware keys, and the user can input operation instructions to the user terminal 20 using the hardware keys. A camera 202 captures an image in response to an imaging instruction from a user. Photographs captured by the camera 202 are stored in a predetermined area of the HDD 210. Further, information from a QR Code® read by the camera 202 can be acquired using a program capable of analyzing QR Codes®. The user terminal 20 can transmit and receive data to and from various kinds of peripheral devices via a Near Field Communication (NFC) communication unit 203, a Bluetooth® communication unit 204, and a wireless Local Area Network (LAN) communication unit 205. The Bluetooth® communication unit 204 of the user terminal 20 may be compatible with Bluetooth® Low Energy.

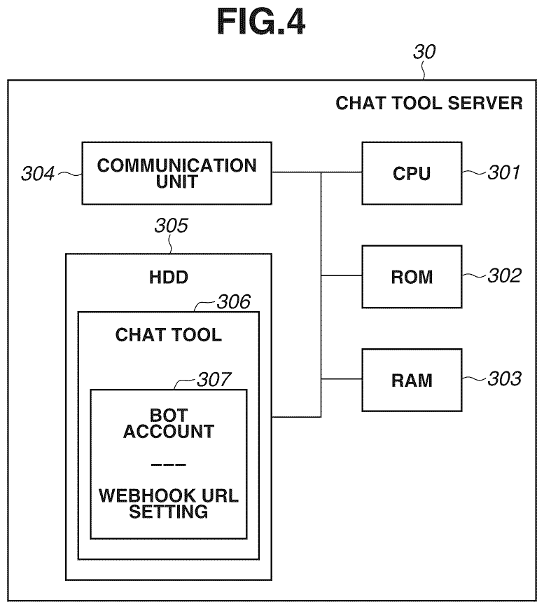

FIG. 4 is a diagram illustrating an example of a hardware configuration of the chat tool server 30.

A CPU 301 reads control programs stored in a ROM 302 and the chat tool 306 stored in an HDD 305, and executes various processes for controlling the operation of the chat tool server 30. The ROM 302 stores the control programs. The RAM 303 is used as a temporary storage area, such as a main memory or a working area of the CPU 301.

The HDD 305 stores various kinds of data, such as messages, images, channel information, and applications. Data can be exchanged with various kinds of devices, such as the user terminal 20 and the image processing apparatus 10, via the communication unit 304. Further, the communication unit 304 may perform wired communication using Ethernet or wireless communication, such as Wi-Fi®. The chat tool 306 is installed on the HDD 305 and operates on the CPU 301. The chat tool server 30 provides bot services in the chat tool 306, and can issue accounts for general users, corporate users, or bots to create a bot account 307. The bot account 307 can perform an automatic response process of responding to a message including a specific keyword with a predetermined message, and a webhook process of hooking a received message to a set URL via HTTP communication. In order to use bot services, a user sets a message of the automatic response process in the bot account 307 or sets a webhook URL of the webhook process.

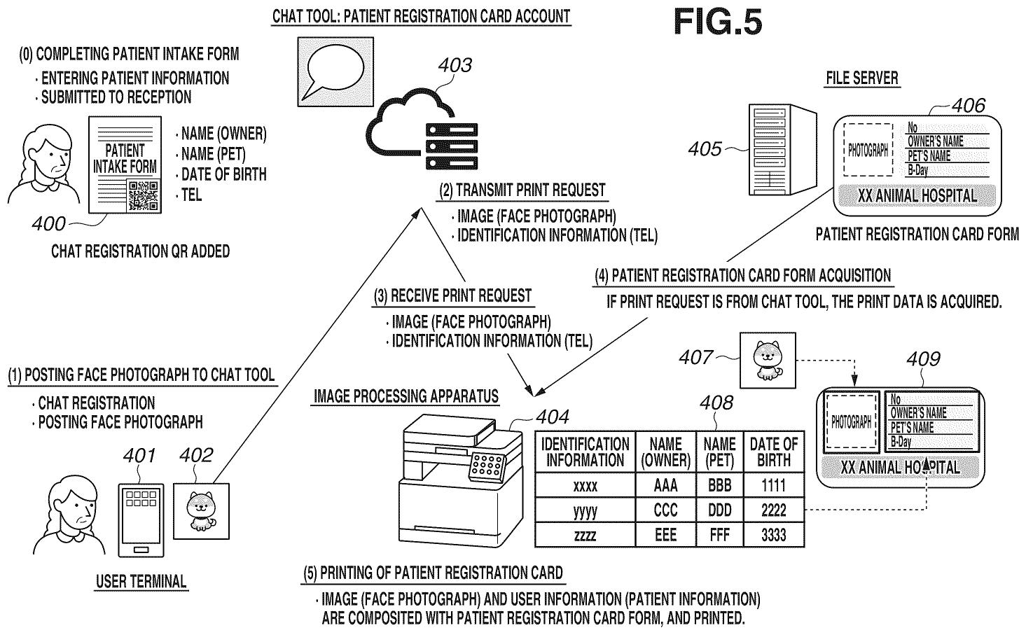

FIG. 5 is a diagram illustrating an example of a process of image compositing and printing from a chat tool.

A patient intake form 400 is handed out at the reception desk and the patient (owner) fills in the owner's name, the pet's name, the date of birth of the pet, a telephone number, and other information. The patient intake form 400 may include a two dimensional code (e.g., QR Code®) for chat registration.

The patient (owner) accesses a chat tool 403 using a user terminal 401 as the user terminal 20 to perform a registration process, and posts image data on a photograph 402 of the pet in the chat. The patient (owner) may enter the pet's name, a telephone number, and the date of birth of the pet here in the chat.

When the image data on the photograph 402 of the pet is posted, the chat tool 403 on the chat tool server 30 transmits a print request for the image to an image processing apparatus 404 as the image processing apparatus 10 registered in advance. In this case, the print request includes the image data received from the patient (owner) and identification information, such as a telephone number associated with the account of the patient (owner) on the chat service. When the patient (owner) enters the pet's name and a telephone number and the date of birth of the pet in the chat, the chat tool 403 transmits the input character strings of the telephone number, and the date of birth, and other information, and the image data to the image processing apparatus 404. In this case, the identification information associated with the account of the patient (owner) on the chat service, the character strings, and the image data may be transmitted, or the character strings and the image data may be transmitted without transmitting the identification information associated with the account of the patient (owner) on the chat service.

When the image processing apparatus 404 receives the image print request from the chat tool 403, the image processing apparatus 404 determines to execute the patient registration card printing, and acquires patient registration card form data 406 from a file server 405.

The image processing apparatus 404 composites the image data (a photograph 407) received via the print request at a predetermined position in the patient registration card form data 406, and outputs print data.

The image processing apparatus 404 also composites patient information to be entered on the patient registration card with the patient registration card form data 406. The patient information (owner's name, pet name, pet date of birth, etc.) composited at this time may be character strings posted in the chat by the patient. Further, when the print request includes identification information associated with the account on the chat service, the patient information to be composited may be the corresponding identification information among the patient information stored in the image processing apparatus 404. The image processing apparatus 404 prints print data 409 generated through the composition. The patient information may be stored in an external server in association with the identification information, instead of the image processing apparatus 10. In this case, the image processing apparatus 10 may transmit the identification information to an external device, and the external device may transmit patient information corresponding to the identification information to the image processing apparatus 10.

According to the above description, the image processing apparatus 404 has an output unit configured to output print data obtained by compositing image data received by a receiving unit and information corresponding to identification information with a form image, and the receiving unit is configured to receive the image data posted on a chat service and the identification information on a user who has posted the image data.

The image processing apparatus further includes a printing unit configured to print an image on a sheet based on the output print data.

The identification information and the patient information on the user may be stored in association with each other by the image processing apparatus 10 or in an external device. If the patient information is stored in the external device, the image processing apparatus 10 receives the patient information corresponding to the identification information on the user from the external device.

The identification information and the patient information on the user may be stored in the image processing apparatus 10 or an external server by a staff member of a hospital inputting the pieces of the information, or may not be stored. For example, the CPU 111 may execute a character recognition process on image data generated by the image processing apparatus 10 scanning the patient intake form 400 using the reading unit 118 to acquire character information. Then, the name (owner) or the name (pet) of the character information may be stored as patient information, and the telephone number of the character information may be stored as identification information on the user in association with the patient information. The character information is not limited to the information acquired by the character recognition process, and may be acquired by a QR Code® (including patient information and identification information) being detected in the document and decoded.

Specifically, according to the above description, the image processing apparatus 10 can composite received image data and information acquired from image data generated by the reading unit reading a document with a form image and output print data. The information acquired from the image data is acquired by the character recognition process being executed on the image data.

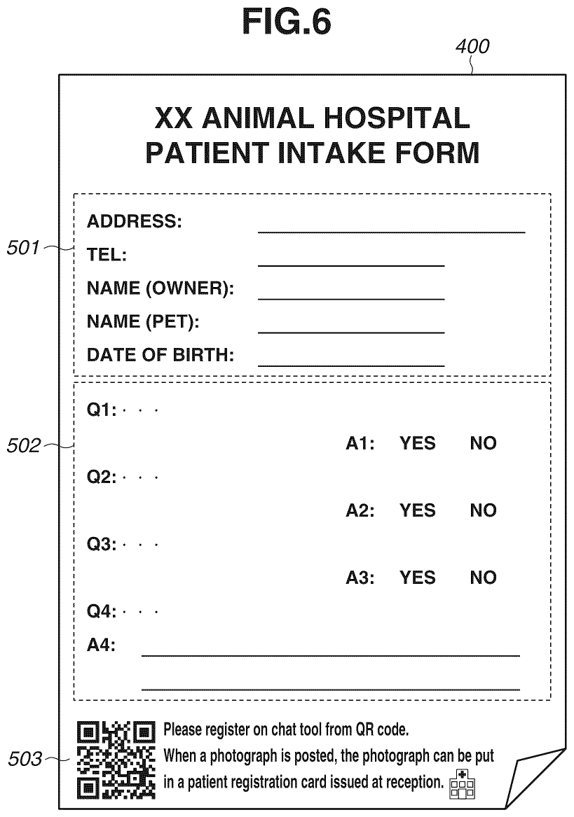

FIG. 6 is a diagram illustrating an example of the patient intake form 400, and details of the patient intake form 400 illustrated in FIG. 5 will be described.

A user information field 501 is a field for entering personal information on a patient and a pet, and includes items, such as an address, a telephone number (TEL), a name (owner), a name (pet), and the date of birth of a pet. A symptom checklist field 502 is a field for entering contents to be informed to a doctor about symptoms of a pet.

Such a patient intake form in a fixed format is used in hospitals, and the size of the patient intake form 400 (e.g., A4), the position of the user information field 501, and the position of each item in the user information field 501 are fixed. A QR Code® 503 includes information (URL) for accessing the bot account 307 of a hospital account issued by the chat tool server 30. The patient can read the QR Code® 503 using the camera 202 of the user terminal 20 to access the bot account 307, and perform a user registration for the bot account 307 as a chat communication destination of the terminal account 212 of the user terminal 20.

When the patient fills in the patient intake form 400 and submits the patient intake form 400 to a hospital clerk, the hospital clerk checks the contents that are filled in and stores data obtained by scanning the patient intake form 400 in the file server 13 as evidence, so that the medical accounting system 11 and the office PC 12 can refer to the scanned patient intake form 400.

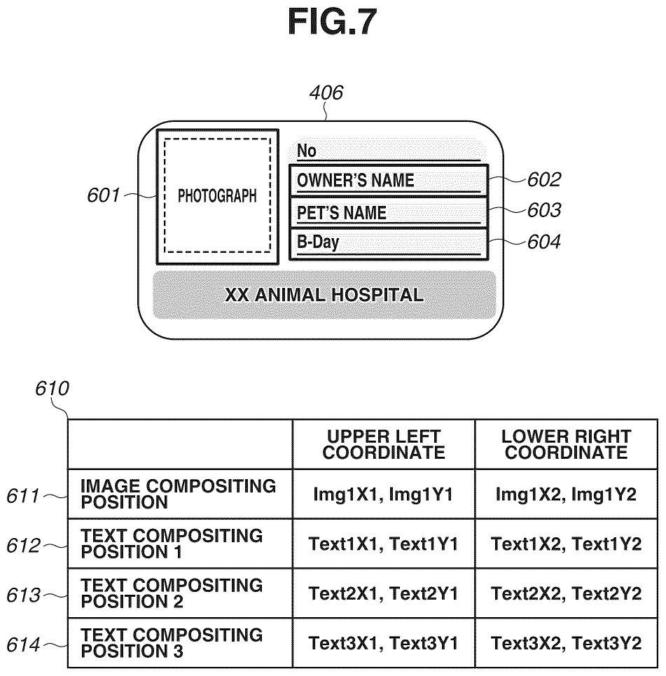

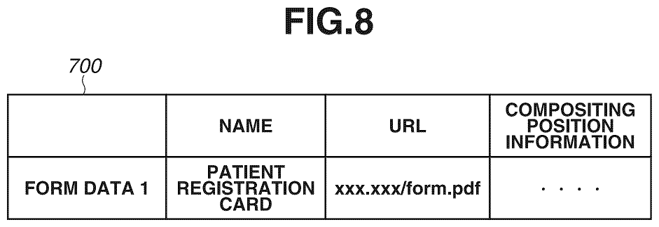

FIG. 7 is a diagram illustrating an example of a patient registration card form. The patient registration card form data 406 illustrated in FIG. 5 will be described in detail. The patient registration card form data 406 is data in which contents to be printed on a patient registration card of a hospital are included, and is document data in formats, such as Portable Document Format (PDF), created by document applications on a PC, or image data in formats, such as Joint Photographic Experts Group (JPEG). The patient registration card form data 406 is also template document data not including contents different between patients, such as a face photograph and user information to be entered in the user information field 501 of the patient intake form 400 on a patient registration card. The patient registration card form data 406 is stored in the file server 13 in advance by a hospital clerk, and the storage location is registered in form data information for the image processing apparatus 10 described below with reference to FIG. 8. The patient registration card form data 406 may be stored in the storage 114 of the image processing apparatus 10 instead of the file server 13.

In the patient registration card form data 406, positions where a face photograph and pieces of user information are arranged are determined in advance. Data is created in which image data on a face photograph prepared separately and text data on user information (or image data obtained by converting the text data into an image) are composited at corresponding positions, and then a patient registration card with the face photograph thereon is output.

An image compositing area 601 is a position where a face photograph is arranged. A text compositing area 1 602 is a position where a name (owner) of user information is arranged. A text compositing area 2 603 is a position where a name (pet) of the user information is arranged. A text compositing area 3 604 is a position where the date of birth of the pet is arranged.

Compositing position information 610 indicates coordinates (top left origin in millimeters) of the compositing areas in the patient registration card form data 406, and is represented by the top-left and bottom-right coordinates of each compositing area.

An image compositing position 611 is coordinates of the image compositing area 601, a text compositing position 1 612 is coordinates of the text compositing area 1 602, a text compositing position 2 613 is coordinates of the text compositing area 2 603, and the text compositing position 3 614 is coordinates of the text compositing area 3 604.

FIG. 8 is a diagram illustrating an example of form data information. As described with reference to FIG. 2, the image processing apparatus 10 has the form compositing and printing function. In the form compositing and printing function, the image processing apparatus 10 stores form data information 700 in the ROM 112 or the storage 114 in order to designate form data and compositing positions with respect to the form data. The form data information 700 can be set and registered by a user who uses the image processing apparatus 10, and is input from a setting screen of a local user interface (LUI) displayed on the operation unit 116. Otherwise, the user inputs the form data information 700 via a setting screen of a remote UI (RUI) displayed on a browser of an external device, such as a PC, via the wireless communication unit 122 or the communication unit 124.

The form data information 700 includes a name, a URL indicating an acquisition location of form data, and compositing position information indicating a compositing position of an image or text. The compositing position information is similar to the compositing position information 610 described with reference to FIG. 7. In the present exemplary embodiment, the URL is described by taking an external device via the network 14, such as the file server 13, as an example, but may be the ROM 112 or the storage 114 in the image processing apparatus 10. Further, a plurality of pieces of form data information 700 may be registered, but in the present exemplary embodiment, form data 1 is associated with the function of the patient registration card printing.

An example of controlling an image size in the compositing and printing function will be described. The image processing unit 126 of the image processing apparatus 10 renders and decodes form data (patient registration card form) and a composite image (face photograph) to convert the form data and the composite image into a raster format. The composite image is subjected to a scaling process in accordance with the size of the image compositing area 601 (the scaling is performed so that either the length between the top and bottom or the width between the left and right matches the frame size, and both the length between the top and bottom and the width between the left and right are equal to the frame size or smaller). The scaled composite image is superimposed on the form data so as to be arranged at the center of the image compositing area 601. Text information 1 (name: an owner) is rendered to generate a text image 1. The text image 1 is subjected to a scaling process in accordance with the size of the text compositing area 1 602. The scaling method is similar to that for the composite image. The scaled image is superimposed on the form data so as to be arranged at the center of the text compositing area 1 602. Text information 2 (name: a pet) and text information 3 (date of birth) are composited in the text compositing area 2 and the text compositing area 3, respectively, in a similar manner to that for the text information 1.

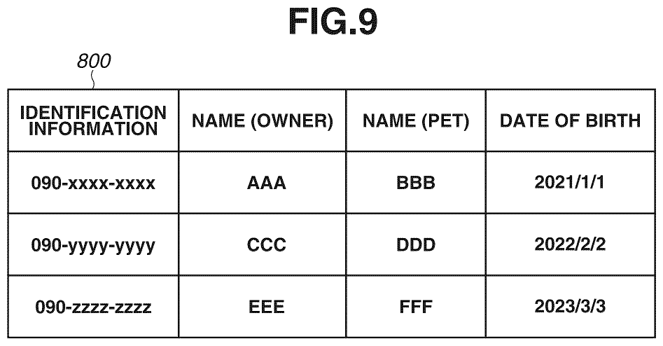

FIG. 9 is a diagram illustrating association between identification information and user information. User management information 800 indicates user management information 408 illustrated in FIG. 5, and here, user information on three persons is registered. As described in FIG. 5, when the image processing apparatus 10 performs an image compositing and printing on the patient registration card form data 406 with identification information (a telephone number, etc.) included in the print request, the image processing apparatus 10 composites corresponding user information at a predetermined position. The user management information 800 in which the identification information for the above purpose is associated with the user information is stored in the ROM 112 or the storage 114. This information is set in the image processing apparatus 10 by a hospital clerk based on a patient intake form submitted by a patient (owner). The hospital clerk inputs this information from the setting screen of the LUI displayed on the operation unit 116 or from a setting screen of an RUI displayed on a browser of an external device, such as a PC, via the wireless communication unit 122 or the communication unit 124.

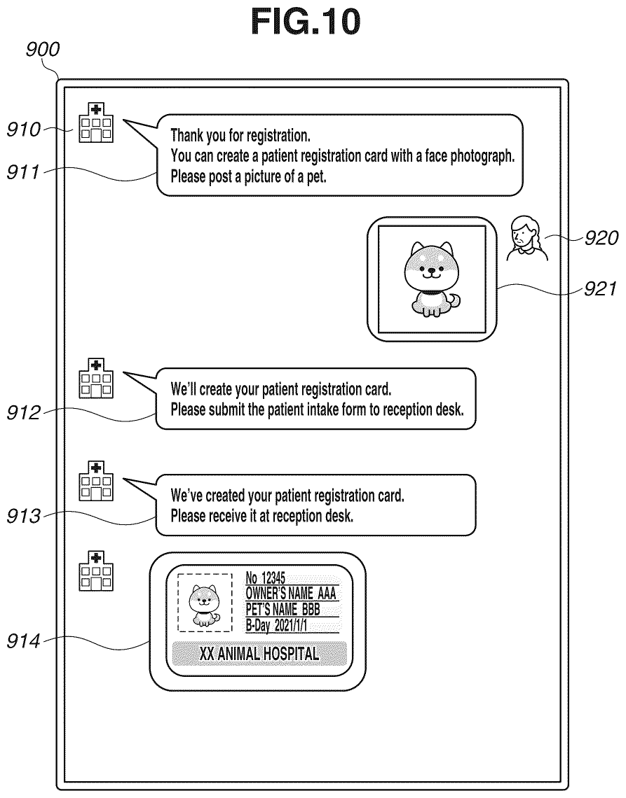

FIG. 10 is a diagram illustrating an example of chat messages. A screen 900 is displayed on the operation panel 201 of the user terminal 20, and displays contents of messages posted to the terminal account 212 of the chat tool 211. An icon 910 is registered in association with the bot account 307 of the chat tool server 30.

An icon 920 is registered in association with a user of the user terminal 20. The patient reads the QR Code® 503 on the patient intake form 400 with the camera 202 of the user terminal 20 and accesses the bot account 307. Then, when the patient performs a user registration for the bot account 307 as a chat communication destination of the terminal account 212 of the user terminal 20, a message 911 is transmitted and displayed by an automatic response function of the bot account 307. Next, when the patient posts a pet photograph 921 according to the message 911, the bot account 307 transmits the image to the image processing apparatus 10 to notify the image processing apparatus 10 of a request for printing a patient registration card, and transmits a message 912 to the terminal account 212. Upon receiving a completion notification of the printing of the patient registration card from the image processing apparatus 10, the bot account 307 transmits a message 913 to the terminal account 212. Upon receiving image data on the patient registration card in which the pet photograph 921 and user information on the patient are composited, from the image processing apparatus 10, the bot account 307 transmits a patient registration card image 914 to the terminal account 212.

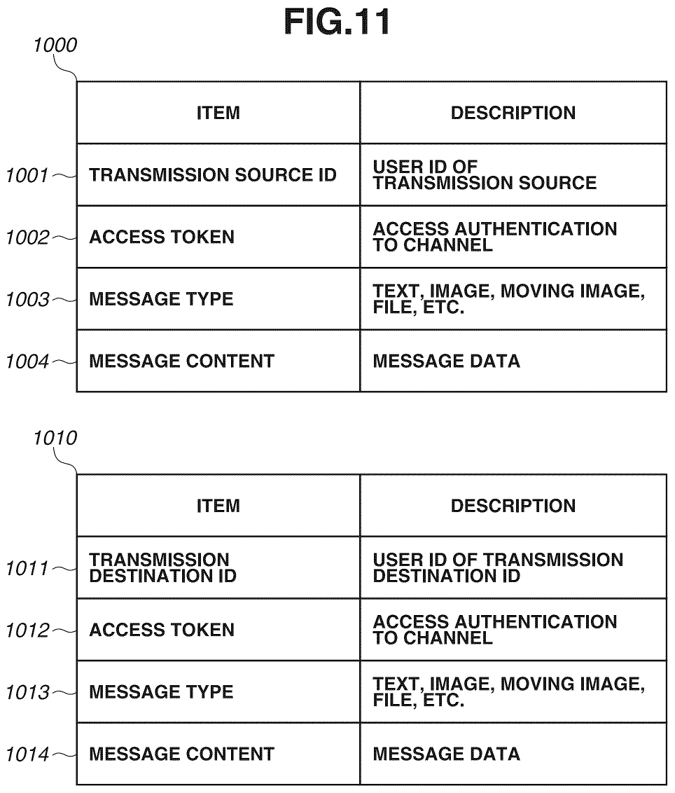

FIG. 11 is a diagram illustrating an example of message events in the chat tool server 30 and the image processing apparatus 10.

A message event 1000 is a table indicating contents of message events to be exchanged with the image processing apparatus 10 by a message Application Programming Interface (API) provided by the chat tool server 30. The bot account 307 of the chat tool server 30 hooks a message transmitted from the terminal account 212 of the user terminal 20 and transmits a message event to a set webhook URL. The image processing apparatus 10 set as a URL interprets the message event using the chat tool control unit 125 and transmits a message event for the response to the chat tool server 30. The message event 1000 is a table indicating message events that are transmitted from the chat tool server 30 to the image processing apparatus 10. A message event 1010 is a table indicating message events that are transmitted from the image processing apparatus 10 to the chat tool server 30. A transmission source identification (ID) 1001 is used to identify the terminal account 212 of the user terminal 20. Access tokens 1002 and 1012 are used for access authentication for communicating with the bot account 307. Message types 1003 and 1013 indicate types of data to be transmitted in message events, and a text is set for a normal text message, and an image is set for an image posting message. In the present exemplary embodiment, a text or image message is used in the description, but other types, such as a moving image or a file, may be used. Message contents 1004 and 1014 are text data and image transmission data corresponding to the types set in the message types 1003 and 1013. In addition, the message APIs provided by the chat tool server 30 include various message events other than the above message events, and for example, includes a profile acquisition event for acquiring profile information on a designated user ID.

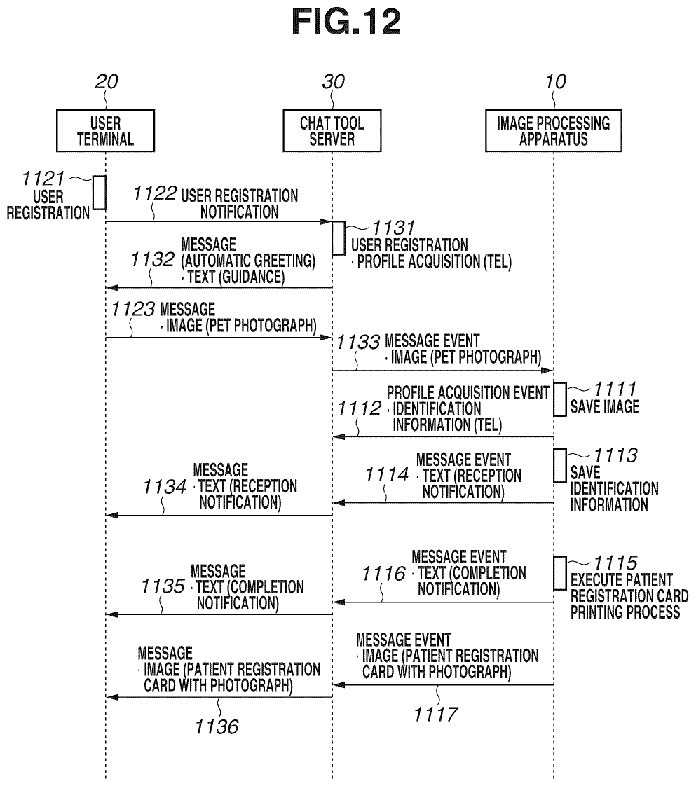

FIG. 12 is a sequence diagram illustrating an example of the image compositing and printing from a chat tool. In the description herein, the terminal account 212 of the user terminal 20 is abbreviated as the user terminal 20, and the bot account 307 of the chat tool server 30 is abbreviated as the chat tool server 30.

In step S1121, the user terminal 20 performs a user registration for the bot account 307, and, in step S1122, performs a user registration notification to the chat tool server 30. In step S1131, in response to that, the chat tool server 30 performs a user registration for the terminal account 212 and acquires profile information (including a telephone number). In step S1132, the chat tool server 30 transmits the message 911 as an automatic response preset for user registration to the user terminal 20.

In step S1123, the photograph 921 is posted from the user terminal 20 to the chat tool server 30, and then in step S1133, the chat tool server 30 hooks a message to the image processing apparatus 10 and transmits image data on the photograph 921 with a message event. In step S1111, in response to that, the image processing apparatus 10 stores the received image data in the storage 114. In step S1112, a profile acquisition event is transmitted to the chat tool server 30, and then in step S1113, profile information on the transmission source ID is acquired and stored in the ROM 112 or the RAM 113.

The profile information also includes a telephone number to be used as identification information to be associated with user information in printing a patient registration card. In step S1114, the image processing apparatus 10 transmits a message event to the chat tool server 30 so that the image processing apparatus 10 transmits the message 912 indicating that the image processing apparatus 10 has accepted the printing of the patient registration card, to the transmission source ID. In step S1134, in response to that, the chat tool server 30 transmits the message 912 to the user terminal 20. In step S1115, the image processing apparatus 10 executes the printing of the patient registration card, and in step S1116, in response to the completion of the printing, the image processing apparatus 10 transmits a message event to the chat tool server 30 so that the message 913 indicating that the printing of the patient registration card is completed is transmitted to the transmission source ID of step S1113. That is, here, when the printing is completed, the image processing apparatus 10 transmits a request for posting information for prompting the user to receive the printed product to the chat tool server 30. In step S1135, in response to that, the chat tool server 30 transmits the message 913 to the user terminal 20.

In step S1117, the image processing apparatus 10 transmits a message event to the chat tool server 30 so that the chat tool server 30 transmits image data on the printed patient registration card to the transmission source ID. In step S1136, in response to that, the chat tool server 30 transmits the patient registration card image 914 to the user terminal 20.

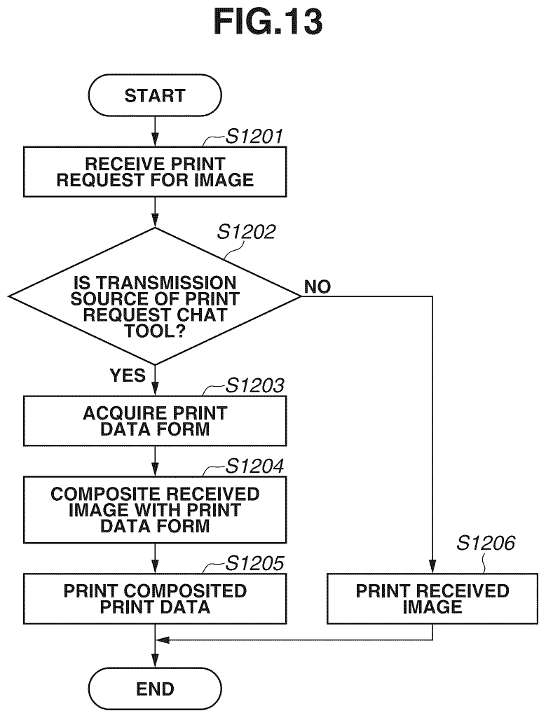

FIG. 13 is a flowchart illustrating an example of the image compositing and printing process from a chat tool. The flow of FIG. 13 is started by the power of the image processing apparatus 10 being turned on.

Each process of the flow of FIG. 13 is executed by the CPU 111 of the image processing apparatus 10 executing programs loaded to the RAM 113.

In step S1201, the image processing apparatus 10 receives a print request for an image, and the chat tool control unit 125 analyzes the print request. In step S1202, if the transmission source of the print request is the chat tool server 30 (YES in step S1202), the processing proceeds to step S1203. In step S1203, the data on the patient registration card form is acquired from an external device or the image processing apparatus 10. In step S1204, the image processing apparatus 10 composites the image received via the print request with print data on the patient registration card form, and in step S1205, the image processing apparatus prints the composited print data. In addition, if the transmission source of the print request is not the chat tool server 30 (NO in step S1202), the processing proceeds to step S1206. In step S1206, the image processing apparatus 10 prints the received image.

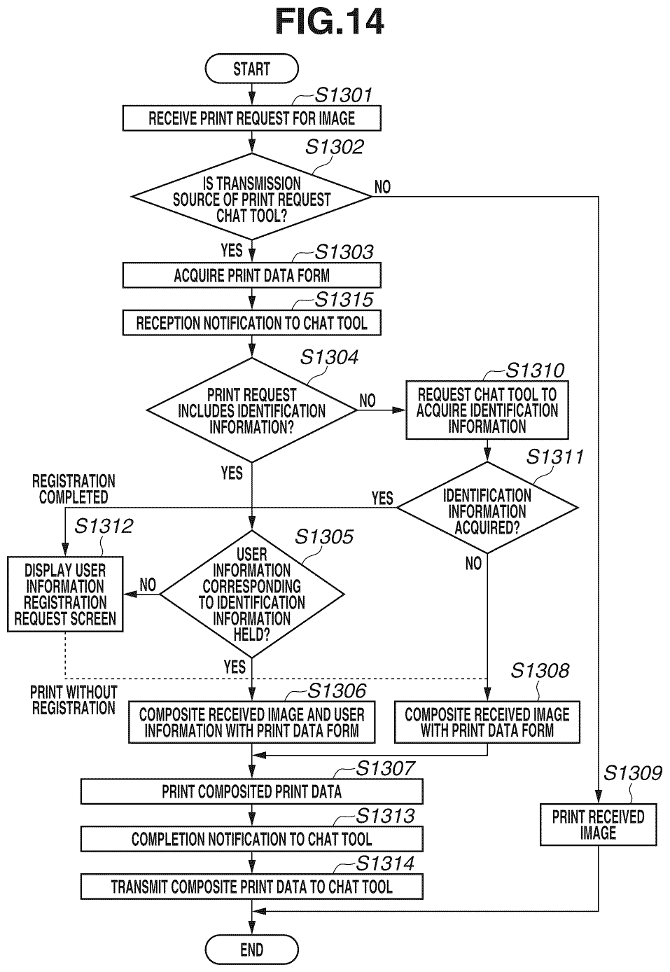

FIG. 14 is a flowchart illustrating an example of the image compositing and printing process from a chat tool. The flow of FIG. 14 is started by the power of the image processing apparatus 10 being turned on.

Each process of the flow of FIG. 14 is executed by the CPU 111 of the image processing apparatus 10 executing programs loaded to the RAM 113. The flow of FIG. 14 is a flow of another mode of the flow of FIG. 13.

In step S1301, the image processing apparatus 10 receives a print request for an image, and the chat tool control unit 125 analyzes the print request. In step S1302, if the transmission source of the print request is a chat tool (YES in step S1302), the processing proceeds to step S1303. In step S1303, the image processing apparatus 10 acquires print data on the patient registration card form from an external device or the image processing apparatus 10. In step S1315, the image processing apparatus 10 transmits a message event as a reception notification to the chat tool server 30. If the transmission source is not a chat tool (NO in step S1302), the processing proceeds to step S1309. In step S1309, the image processing apparatus 10 prints the image on a sheet based on the received image data.

In step S1304, if identification information is included in the print request (YES in step S1304), the processing proceeds to step S1305. In step S1305, the image processing apparatus 10 refers to the user management information 800 stored in the image processing apparatus 10 and determines whether there is user information corresponding to the identification information. In step S1306, the received image corresponding to the identification information and the user information are composited with the print data on the patient registration card form.

In step S1304, if identification information is not included in the print request (NO in step S1304), the processing proceeds to step S1310. In step S1310, the image processing apparatus 10 transmits a profile acquisition event to the chat tool server 30 to request the chat tool server 30 to acquire identification information. In step S1311, if the identification information is acquired (YES in step S1311), the process proceeds to step S1312. If not acquired (NO in step S1311), the processing proceeds to step S1308. The processing in steps S1308, S1307, and S1309 are similar to those in steps S1204, S1205, and S1206 in FIG. 12.

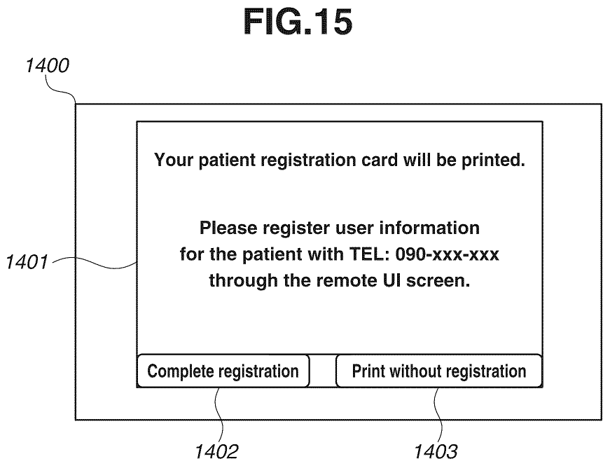

In step S1305, if the image processing apparatus 10 does not hold user information corresponding to the identification information (NO in step S1305), the processing proceeds to step S1312. In step S1312, the image processing apparatus 10 displays a user information registration request screen 1400 in FIG. 15. A screen 1401 displays identification information (a telephone number) and a message prompting the user to register user information from the remote UI. When a button 1402 is pressed, it is determined that the registration of user information is completed, and the process returns to step S1305. When a button 1403 is pressed, it is determined that only the received image is to be composited with the patient registration card form and printed without registering user information, and the processing proceeds to step S1308. The remote UI is a setting screen displayed by the user terminal 20 accessing a web server that the image processing apparatus 10 serves as, using a web browser.

In step S1307, the image processing apparatus 10 completes the printing of the composited print data, and then in step S1313, the image processing apparatus 10 transmits a message event as a completion notification to the chat tool server 30. In step S1314, the image processing apparatus 10 transmits the composited and printed image data to the chat tool server 30 with a message event.

The above process can reduce the time and effort to create print data in which image data is composited with a form.

### Other Embodiments

Embodiment(s) of the present disclosure can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc™ (BD)), a flash memory device, a memory card, and the like.

While the present disclosure has described exemplary embodiments, it is to be understood that some embodiments are not limited to the disclosed exemplary embodiments. The scope of the following claims is to be accorded the broadest interpretation so as to encompass all such modifications and equivalent structures and functions.

This application claims priority to Japanese Patent Application No. 2024-043877, which was filed on Mar. 19, 2024 and which is hereby incorporated by reference herein in its entirety.

What is claimed is:

1. An image forming apparatus comprising:
a printer;
at least one processor; and
at least one memory,
wherein the at least one memory stores instructions for causing the at least one processor and the at least one memory to:
receive image data posted to a chat service and identification information on a user who has posted the image data; and
output print data by compositing the received image data and information corresponding to the identification information with a form image, and
wherein the printer is configured to print an image on a sheet based on the output print data.

2. The image forming apparatus according to claim 1, further comprising a storage device configured to store the identification information and the information in association with each other,
wherein the at least one memory stores instructions for causing the at least one processor and the at least one memory to output the print data by compositing the received image data and the information corresponding to the identification information, the information corresponding to the identification information being stored in the storage device, with the form image.

3. The image forming apparatus according to claim 1, wherein the at least one memory stores instructions for causing the at least one processor and the at least one memory to
receive the information corresponding to the identification information, and
output the print data by compositing the image data and the received information corresponding to the identification information with the form image.

4. The image forming apparatus according to claim 1, wherein the image data and the identification information are received from a chat server configured to provide the chat service.

5. The image forming apparatus according to claim 1, further comprising a scanner configured to read an image of a document to generate image data,
wherein the information corresponding to the identification information is information obtained from the image data generated by the scanner reading the document.

6. The image forming apparatus according to claim 5, wherein the information corresponding to the identification information is information obtained by executing a character recognition process on the image data generated by the scanner reading the document.

7. The image forming apparatus according to claim 1, wherein the identification information is a telephone number of the user.

8. The image forming apparatus according to claim 7, wherein the identification information is a telephone number registered in association with an account of the user registered in the chat service.

9. The image forming apparatus according to claim 1, wherein the at least one memory stores instructions for causing the at least one processor and the at least one memory are further configured to, in response to a completion of printing of the image based on the print data by the printer, transmit a request for posting information for prompting the user to receive a printed product, to the chat service.

10. An image forming method comprising:
receiving image data posted to a chat service and identification information on a user who has posted the image data;
outputting print data by compositing the received image data and information corresponding to the identification information with a form image; and
printing an image on a sheet based on the output print data.

11. A non-transitory computer readable storage medium including computer-executable instructions for causing an image forming apparatus to perform an image forming method, the image forming method comprising:
receiving image data posted to a chat service and identification information on a user who has posted the image data;
outputting print data by compositing the received image data and information corresponding to the identification information with a form image; and
printing an image on a sheet based on the output print data.
